(19) United States
(12) Patent Application Publication
(10) Pub. No.: US 2025/0135186 A1
(43) Pub. Date: May 1, 2025

(54) BLOOD PUMP WITH SURFACE MODIFICATION

(71) Applicant: ABIOMED, Inc., Danvers, MA (US)

(72) Inventors: Smriti Singh, Aachen (DE); Claudia Mourran, Aachen (DE)

(73) Assignee: ABIOMED, Inc., Danvers, MA (US)

(21) Appl. No.: 18/931,258

(22) Filed: Oct. 30, 2024

Related U.S. Application Data
(60) Provisional application No. 63/546,883, filed on Nov. 1, 2023.

(51) Int. Cl. A61M 60/90; A61M 60/13; A61M 60/81

(52) U.S. Cl. CPC A61M 60/90; A61M 60/13; A61M 60/81; A61M 2205/0216; A61M 2205/0238; A61M 2205/025; A61M 2205/103; A61M 2205/75; A61M 2206/20

(57) ABSTRACT

A blood pump may be provided. The blood pump may include a blood flow section operably coupled to a distal end of a catheter. The blood flow section may be configured to cause blood to flow into a blood flow inlet of the blood flow section, through the blood flow section, and out of a blood flow outlet. At least one external and/or internal surface of the blood flow section may include at least a portion of a surface modified by one or more fluorinated end groups and/or silicone end groups of an oligomer or copolymer. The surface may be modified by one or more fluorinated end groups of an oligomer. The surface may be modified by one or more silicone end groups of a copolymer. The oligomer or copolymer may include a silicone, a polycarbonate, a polyurethane, a polyamide, a polyethylene, a polypropylene, a polysulfone, or a polyvinyl chloride.

### CROSS-REFERENCE TO RELATED APPLICATIONS

The present application claims priority to U.S. Provisional Application No. 63/546,883 filed Nov. 1, 2023, which is incorporated by reference herein by its entirety.

### TECHNICAL FIELD

The present disclosure is drawn to blood pumps, and specifically, blood pumps with modifications to surfaces through or along which blood flows.

### BACKGROUND

Blood pumps are used to provide a variety of therapies and treatments for patients. Such pumps may be, e.g., inserted into a blood vessel, and may be configured to cause blood to flow from one location to another in the body. Such devices must be designed to minimize regions of stagnant blood flow, to avoid creating conditions that can cause thrombosis.

### BRIEF SUMMARY

In various aspects, a blood pump may be provided. The blood pump may include a blood flow section operably coupled to a distal end of a catheter. The blood flow section may be configured to cause blood to flow into a blood flow inlet of the blood flow section, through the blood flow section, and out of a blood flow outlet. At least one external and/or internal surface of the blood flow section may include at least a portion of a surface modified by one or more fluorinated end groups and/or silicone end groups of an oligomer or copolymer.

In some embodiments, at least a portion of the surface may be modified by one or more fluorinated end groups of an oligomer. At least a portion of the surface may be modified by one or more silicone end groups of a copolymer. The oligomer or copolymer may comprise a silicone, a polycarbonate, a polyurethane, a polyamide, a polyethylene, a polypropylene, a polysulfone, or a polyvinyl chloride. In some embodiments, a portion of both an internal surface and an external surface may be modified by one or more fluorinated end groups and/or silicone end groups. In some embodiments, all of an external surface and/or an internal surface of the blood flow sections may be modified by one or more fluorinated end groups and/or silicone end groups of an oligomer or copolymer.

The surface modification may cause the surface to have a lower coefficient of friction compared to a coefficient of friction of the surface without the one or more fluorinated end groups and/or silicone end groups of an oligomer or copolymer. The surface modification may cause the surface to have a lower thrombogenicity compared to a thrombogenicity of the surface without the one or more fluorinated end groups and/or silicone end groups of an oligomer or copolymer. The surface modification may cause the surface to have less biofouling compared to the biofouling of the surface without the one or more fluorinated end groups and/or silicone end groups of an oligomer or copolymer.

Some or all of the surfaces may be modified. In some embodiments, a portion of both an internal surface and an external surface may be modified by one or more fluorinated end groups and/or silicone end groups of an oligomer or copolymer. In some embodiments, all of the external and/or internal surface of the blood flow section may be surface modified by one or more fluorinated end groups and/or silicone end groups of an oligomer or copolymer.

Different surfaces, or different portions of the surfaces, may be modified differently. A first portion of an external and/or internal surface of the blood flow section may be surface modified by one or more first fluorinated end groups and/or silicone end groups of a first oligomer or copolymer. A second portion of an external and/or internal surface of the blood flow section may be surface modified by one or more second fluorinated end groups and/or silicone end groups of a second oligomer or copolymer where the first portion and second portions are different portions. The first fluorinated end groups and/or silicone end groups may be different from the second fluorinated end groups and/or silicone end groups. The first oligomer or copolymer may be different from the second oligomer or copolymer.

In some embodiments, at least a portion of the blood flow section may be expandable. The blood flow section may include an expandable pump housing. The expandable pump housing may include at least one strut. The at least one strut may be a helical strut. The helical strut may be configured to form a plurality of geometric apertures. The at least one strut may be a polymer. The at least one strut may be coupled to a polymer. A polymer may be coupled to an inner surface of the at least one strut. A polymer may be coupled to an outer surface of the at least one strut. The polymer may define an external and/or internal surface of the blood flow section that is at least partially surface modified by the one or more fluorinated end groups and/or silicone end groups of an oligomer or copolymer. The polymer may be disposed on at least one intermediate portion of the expandable pump housing, the intermediate portion starting a distance d1>0 from a distal end of the pump housing and a distance d2>0 from a proximal end of the pump housing. The polymer may form an inner liner preventing blood from flowing radially through the blood flow section in the intermediate portion. The polymer may form an outer liner preventing blood from flowing radially through the blood flow section in the intermediate portion.

In some embodiments, the blood flow section may include an expandable inlet. The expandable inlet may include an inflow funnel in an expandible basket. The inflow funnel may define an external and/or internal surface of the blood flow section that are at least partially surface modified by the one or more fluorinated end groups and/or silicone end groups of an oligomer or copolymer. The expandible basket may be configured to be expanded by a balloon. The expandible basket may include one or more struts. The expandible basket may include a polymer coupled to a portion of the one or more struts to form the inflow funnel, such that, when the expandible basket is in an expanded configuration, the inflow funnel has an open distal end to allow blood to flow into the inflow funnel.

In some embodiments, the blood flow section may include a motor housing coupled to a pump housing, the pump housing defining the blood flow inlet and a pump housing outlet. In some embodiments, the blood flow outlet may be the pump housing outlet. The blood flow section may include an outflow tube coupled to the pump housing. The outflow tube may be configured such that blood may flow out of the pump housing outlet, through the outflow tube, and out an outflow tube outlet, wherein the blood flow outlet is the outflow tube outlet. The outflow tube may define an external and/or internal surface of the blood flow section that is at least partially surface modified by the one or more fluorinated end groups and/or silicone end groups of an oligomer or copolymer.

The blood flow section may include at least one filter placed over the blood flow inlet as a filter sleeve. The filter sleeve may filter blood entering the blood flow inlet. The at least one filter may include a plurality of apertures each having a width of 40 μm to 120 μm. The filter sleeve may include a circumferentially closed filter sleeve. The filter sleeve may include a slit along its length to allow the at least one filter to be placed over the blood pump in a direction traverse to a central axis of the blood pump. The filter sleeve may have a mesh structure. The mesh structure may include elongate slot-like apertures separated by struts. At least a portion of the filter sleeve may include a shape-memory material. The filter sleeve may include a tapered portion at one or both ends of the filter sleeve.

The blood flow section may include a cannula. The cannula may be coupled to a distal end of a motor housing. The motor housing may define the blood flow outlet. The cannula may define an external and/or internal surface of the blood flow section that is at least partially surface modified by the one or more fluorinated end groups and/or silicone end groups of an oligomer or copolymer.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a system.

FIG. 2 is an illustration of a blood pump.

FIG. 3 is a cross-sectional illustration of a blood pump's pump section.

FIG. 4 is a cross-sectional illustration of an expandable pump housing.

FIG. 5 is an illustration of an expandable inlet.

FIG. 6 is an illustration of a blood pump.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

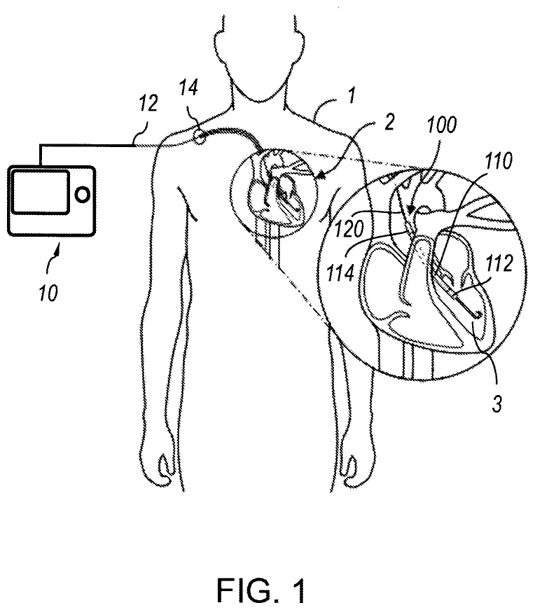

In various aspects, a blood pump may be provided. Referring to FIG. 1, a blood pump (100) may be deployed within a blood vessel of a patient (1), such as in a heart (2). The blood pump may include a blood flow section (110) operably coupled to a distal end of a catheter (120). The blood flow section may be configured to cause blood to flow into a blood flow inlet (112) of the blood flow section, through the blood flow section (110), and out of a blood flow outlet (114). Here, the blood flow inlet is shown as being disposed within a left ventricle 3, but as will be understood, the pump could be deployed at other locations within a patient. The pump may be operably coupled to a controller (10) via, e.g., one or more wires (12) or tubes through an access point (14). The controller (10) may be used to control the blood pump.

At least one external and/or internal surface of the blood flow section may include at least a portion of a surface modified by one or more fluorinated end groups and/or silicone end groups of an oligomer or copolymer. The surface may be modified by one or more fluorinated end groups of an oligomer. The surface may be modified by one or more silicone end groups of a copolymer. The oligomer or copolymer may comprise a silicone, a polycarbonate, a polyurethane, a polyamide, a polyethylene, a polypropylene, a polysulfone, or a polyvinyl chloride.

The surface modification may cause the surface to have a lower coefficient of friction compared to a coefficient of friction of the surface without the one or more fluorinated end groups and/or silicone end groups of an oligomer or copolymer. The surface modification may cause an increase in thermal stability of the polymer used to form the surface (relative to the polymer without the surface modification). This thermal stability increase may comparatively reduce degradation of the polymer during processing (e.g., during laser cutting of the polymer). The surface modification may cause the surface to have a lower thrombogenicity compared to a thrombogenicity of the surface without the one or more fluorinated end groups and/or silicone end groups of an oligomer or copolymer. The surface modification may cause the surface to have less biofouling compared to the biofouling of the surface without the one or more fluorinated end groups and/or silicone end groups of an oligomer or copolymer.

Some or all of the surfaces may be modified. In some embodiments, a portion of both an internal surface and an external surface may be modified by one or more fluorinated end groups and/or silicone end groups of an oligomer or copolymer. In some embodiments, all of the external and/or internal surface of the blood flow section may be surface modified by one or more fluorinated end groups and/or silicone end groups of an oligomer or copolymer.

Different surfaces, or different portions of the surfaces, may be modified differently. A first portion of the external and/or internal surface of the blood flow section may be surface modified by one or more first fluorinated end groups and/or silicone end groups of a first oligomer or copolymer. A second portion of the external and/or internal surface of the blood flow section may be surface modified by one or more second fluorinated end groups and/or silicone end groups of a second oligomer or copolymer where the first portion and second portions are different portions. The first fluorinated end groups and/or silicone end groups may be different from the second fluorinated end groups and/or silicone end groups. The first oligomer or copolymer may be different from the second oligomer or copolymer.

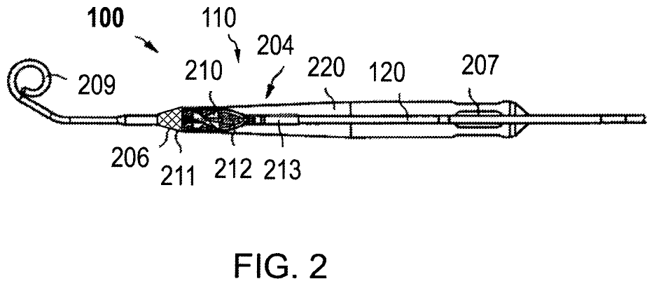

For example, referring to FIG. 2, an embodiment of a blood pump (100) can be seen, having a blood flow section (110) coupled to a distal end of catheter (120). A flexible atraumatic tip (209) may be coupled to a distal end of the blood flow section (110). The blood flow section (110) may include an impeller (210) within pump housing (211) (which may be an expandable pump housing). The impeller (210) may be operably coupled to an electric motor within a motor housing (213) via a rigid drive shaft (e.g., drive shaft (212)). In some embodiments, when the impeller rotates, blood is configured to flow through a blood flow inlet (206), through the pump section, out of the pump section and into outflow tube (220), and out a blood flow outlet (207) (here, an outlet from the outflow tube). Impeller (210) may be a compressible and expandable impeller. For example, impeller (210) may be in a compressed state when pump housing (211) is in a compressed state when the blood flow section (110) of the intravascular blood pump (100) is introduced into a patient's vasculature. In some embodiments, an electric motor housing (213) may be disposed proximal to the impeller (210) and distal to the blood flow outlet (207).

In some embodiments, one or more portions of the inner or outer surfaces of the pump section may have a surface modified by one or more fluorinated end groups and/or silicone end groups of an oligomer or copolymer. In some embodiments, one or more portions of the inner or outer surfaces of the outflow tube section may have a surface modified by one or more fluorinated end groups and/or silicone end groups of an oligomer or copolymer.

The outflow tube is preferably collapsible. The outflow tube may be composed of any suitable biocompatible material, such as a suitable polymer, such as polyurethane, polyamide, nylon, or silicone. In some embodiments, the outflow tube may be polytetrafluoroethylene (PTFE)

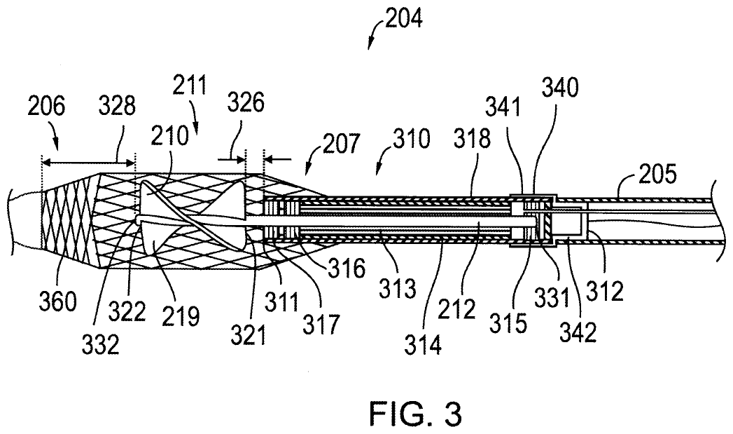

Referring to FIGS. 2 and 3, blood flow section (110) may include a pump section (204). Pump section (204) may include the pump housing (211). In FIG. 2, the pump housing (211) is shown as defining the blood flow inlet and a pump housing outlet (which allows blood to flow into outflow tube (220)). In FIG. 3, the pump housing (211) is shown as defining the blood flow inlet and the blood flow outlet (with no outflow tube shown, the pump housing outlet becomes the blood flow outlet).

Pump section (204) may include a drive section (310). The drive section may have a distal end (311) and a proximal end (312). Drive section (310) may include an electric motor formed from a stator (314) operably coupled to a rotor (313), configured such that the rotational speed of the rotor can be controlled based on electrical properties of electrical current provided to electromagnets of the stator. The rotor (313) and stator (314) may be coaxially aligned.

Drive section (310) may include a plurality of bearings, including one or more proximal bearings (315) and one or more distal bearings (316, 317). In some embodiments, at least one bearing is a radial bearing. In some embodiments, at least one bearing is an axial bearing.

Pump section (204) may include a motor housing (318). Motor housing (318) may be disposed around the electric motor (e.g., the rotor and stator). In some embodiments, the motor housing (318) may be disposed around the entire drive section (310). In some embodiments, the motor housing (318) may be disposed around the rotor (313), the stator (314), and at least one bearing (e.g., a proximal bearing (315) and a distal bearing (316)). One or more of the bearings may include a ceramic bearing (e.g., be at least partially formed of a ceramic material). In some embodiments, one or more bearings may be a full ceramic bearing.

Motor housing (318) may be coupled to a distal end of the catheter. Motor housing (318) may have an outer diameter at a distal end that is no larger than an outer diameter of catheter (205). Motor housing (318) may have an outer diameter at a distal end that is larger than an outer diameter of catheter (205). Motor housing (318) may have an outer diameter at a distal end that is no larger than 12 French (12 F), preferably no larger than 10 French (10 F). Motor housing (318) also may have an outer diameter at a distal end that is no larger than 3.3 mm±0.5 mm. Motor housing (318) also may have an outer diameter at a distal end that is no larger than 4.0 mm±0.5 mm.

In some embodiments, the outer diameter of the motor housing may vary. Motor housing (318) may have an average outer diameter that is no larger than an outer diameter of catheter (205). Motor housing (318) may have an average outer diameter that is larger than an outer diameter of catheter (205). Motor housing (318) may have an average outer diameter that is no larger than 12 French (12 F), preferably no larger than 10 French (10 F). Motor housing (318) also may have an average outer diameter that is no larger than 3.3 mm±0.5 mm. Motor housing (318) also may have an average outer diameter that is no larger than 4.0 mm±0.5 mm.

In some embodiments, motor housing (318) may have a constant outer diameter. In some embodiments, motor housing (318) may have a diameter at the proximal end that is larger than a diameter at a distal end.

Motor housing (318) may be coupled to catheter (205). In some embodiments, a proximal end of motor housing (318) may be disposed within a distal end of catheter (205). In some embodiments, a proximal end of the motor housing (318) may not be disposed within a distal end of the catheter (205). In some embodiments, pump section (204) may include a housing connector (340). Housing connector (340) may have a proximal portion (341) disposed within a distal end of catheter (205), and a distal portion (342) disposed around a proximal end of motor housing (318).

The rotor (313) may be coupled to a drive shaft (212). The drive shaft (212) may be coupled to an impeller (210) disposed within a pump housing (211) (where the pump housing (211) may form a cage around the impeller (210)), the impeller (210) being configured to cause blood to flow from a blood flow inlet (206) at a distal end of the pump section (204) to a blood flow outlet (207) (here, an outlet from the pump housing) located proximally to the blood flow inlet (206). Note, as used herein, the term “impeller” is used to avoid confusion with the “rotor” (313) of the electric motor. However, the term “impeller” as used herein is intended to refer to any rotating component comprising one or more blades (219), where the blades are configured to cause blood to move from the blood flow inlet towards the blood flow outlet when the impeller rotates.

A proximal end (331) of the drive shaft (212) may be disposed within the drive section (310), such as, e.g., within a proximal bearing (315), and the drive shaft (212) may extend distally, extending beyond a distal end (311) of the drive section (310). In some embodiments, the distal end (332) of the drive shaft may extend to the distal end (322) of the impeller (210). In some embodiments, the distal end (332) of the drive shaft may extend distally beyond the distal end (322) of the impeller (210). In some embodiments, there is an axial gap (328) between the distal end (332) of the drive shaft (212) and the distal end of the pump housing (211). In some embodiments, there is an axial gap between the distal end (332) of the drive shaft (212) and the distal end of the blood flow inlet (206). In some embodiments, the drive shaft (212) does not extend into catheter (205).

In some embodiments, there is an axial gap (326) between the proximal end (321) of the impeller (210) and the distal end (311) of the drive section (310).

The drive shaft (212) may be composed of a rigid material. In some embodiments, drive shaft (212) can be composed of a breakproof ceramic, diamond, a stainless steel (such as 1.4441, 316 L, etc.), a cobalt alloy (such as MP35N, 35N LT, etc.), or a combination thereof. The drive shaft (212) may be coated with an amorphous carbon coating (DLC=diamond-like carbon or diamond-like coating). DLC layers may be especially wear-resistant and low-friction. They may be only a few micrometers thick and can be produced for example by chemical vapor deposition (CVD) or physical vapor deposition (PVD). Impeller (210) may be made from a compressible and expandable plastic material. For example, impeller (210) may be directly casted or glued onto drive shaft (212).

In some embodiments, one or more portions of the inner or outer surfaces of the pump section (including the blood flow inlet, the outlet from the pump housing, and/or the intermediate portion around the impeller) as seen in FIG. 3 may have a surface modified by one or more fluorinated end groups and/or silicone end groups of an oligomer or copolymer.

At least a portion of the blood flow section may be expandable. The blood flow section may include, e.g., an expandable pump housing (see, e.g., pump housing (211) in FIGS. 2-4).

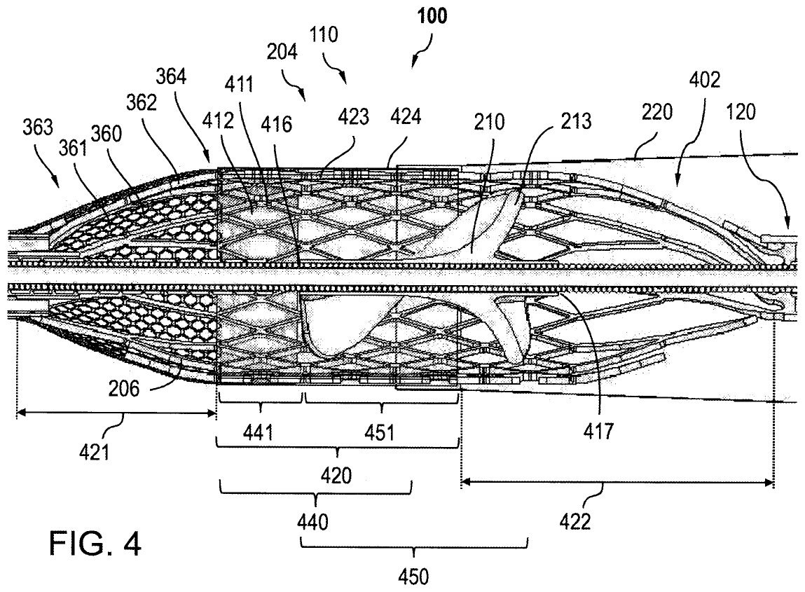

Referring to FIG. 4, an embodiment of a blood pump may be provided. As disclosed herein, the blood pump (100) may include a blood flow section (110) coupled to a catheter (120). Typically, a distal end of a catheter will be coupled to a proximal end of a housing (e.g., pump housing (211)), although other configurations are possible.

The pump housing may include at least one strut, such as a plurality of struts (411). The at least one strut may be a helical strut configured to form a plurality of geometric apertures (412). The at least one strut may be a polymer. The polymer may be disposed on at least an intermediate portion (420) of the expandable pump housing. The intermediate portion may start a distance (421) (“d1”)>0 from a distal end of the pump housing and a distance (422) (“d2”)>0 from a proximal end of the pump housing.

An impeller (210) with at least one blade (219) may be disposed within the pump housing. The rotation of the impeller around a central axis may cause blood to flow, e.g., from a blood flow inlet (206), through the pump housing, to an outlet (207). The at least one blade may have an axial length that is 7.5 mm or less. In some embodiments, the length is at least 6 mm. In some embodiments, the length is at least 5 mm. In some embodiments, the length is at least 4 mm. In some embodiments, the length is at least 3 mm.

The housing may include an inner coating layer (423) and an outer coating layer (424) around the struts. The inner coating may define an inner surface. The inner surface may be a smooth surface. As used herein, the term “smooth surface” is intended to refer to a surface that is free of protrusions, cavities, dimples, vents, or other such deviations that extend from (or into) the surface by more than a certain distance. The distance is preferably no more than 0.5 mm, more preferably no more than 0.25 mm, and still more preferably no more than 0.1 mm.

That is, at least one strut may be coupled to a polymer, such as a polyurethane. A polymer may be coupled to an inner surface of the strut. A polymer may be coupled to an outer surface of the strut. A polymer may be coupled to both an inner and outer surface of the strut. The polymer may define an external and/or internal surface of the blood flow section that is at least partially surface modified by the one or more fluorinated end groups and/or silicone end groups of an oligomer or copolymer. The polymer may form an inner liner preventing blood from flowing radially through the blood flow section in the intermediate portion. The polymer may form an outer liner preventing blood from flowing radially through the blood flow section in the intermediate portion.

The coating may extend from the blood flow inlet towards the blood flow outlet by a fixed distance (e.g., axial length of intermediate portion (420)). An outflow tube (220) may be coupled to the pump housing (211). The outflow tube (220) may be coupled to the outer coating layer. The outflow tube (220) may cover and surround the outlet (402) from the housing. The outflow tube may be composed of a polymer. The outflow tube may define an external and/or internal surface of the blood flow section that is at least partially surface modified by the one or more fluorinated end groups and/or silicone end groups of an oligomer or copolymer.

The impeller may be offset from the blood inlet by a predetermined distance (e.g., an axial distance). The impeller may be positioned such that the leading edge (416) of the impeller is surrounded by the coating layers. The trailing edge (417) of the impeller may also be surrounded by the coating, or may extend beyond the trailing end of the coating. However, if the impeller extends beyond the coating, at least a portion (451) of the total axial length (450) of the impeller is surrounded by the coating layers. In some embodiments, that portion (451) is at least 60% of the length of the impeller (i.e., axial length of portion (451)≥60%× the total axial length (450) of the impeller). In some embodiments, that portion (451) is 50-60% of the length of the impeller (i.e., 50%× the total axial length (450) of the impeller≤axial length of portion (451)≤60%× the total axial length (450) of the impeller).

In some embodiments, the inner coating may have an axial length (e.g., fixed axial distance (440)) configured to surround a portion of the impeller, the portion having an axial length (441) that is 50% or less of a total axial length (450) of the impeller. In some embodiments, the inner coating may have an axial length (e.g., fixed axial distance (440)) configured to surround a portion of the impeller, the portion having an axial length (441) that is 30% or less of a total axial length (450) of the impeller. In some embodiments, the inner coating may have an axial length (e.g., fixed axial distance (440)) configured to surround a portion of the impeller, the portion having an axial length (441) that is 20% or less of a total axial length (450) of the impeller. In some embodiments, the portion may have an axial length (441) that is 15% or less of a total axial length (450) of the impeller. In some embodiments, the portion may have an axial length (441) that is 10% or less of a total axial length (450) of the impeller.

In some embodiments, the inner coating may have an axial length (e.g., fixed axial distance (440)) configured to surround a portion of the at least one blade, the portion having an axial length (441) that is 50% or less of a total axial length (450) of the at least one blade. In some embodiments, the inner coating may have an axial length (e.g., fixed axial distance (440)) configured to surround a portion of the at least one blade, the portion having an axial length (441) that is 30% or less of a total axial length (450) of the at least one blade. In some embodiments, the inner coating may have an axial length (e.g., fixed axial distance (440)) configured to surround a portion of the at least one blade, the portion having an axial length (441) that is 20% or less of a total axial length (450) of the at least one blade. In some embodiments, the portion may have an axial length (441) that is 15% or less of a total axial length (450) of the at least one blade. In some embodiments, the portion may have an axial length (441) that is 10% or less of a total axial length (450) of the at least one blade.

The inner coating may be disposed on the inner surface at the blood inlet and extending axially partially towards the blood outlet for a fixed axial distance (440) that is less than an axial length of the housing. That is, the inner coating may not extend the entire axial distance between the blood inlet and the blood outlet.

The blood flow section may include at least one filter (360). The filter(s) may be placed over the blood flow inlet as a filter sleeve. The filter sleeve may filter blood entering the blood flow inlet. The at least one filter may include a plurality of apertures (361) each having a width of 40 μm to 120 μm. The filter sleeve may include a circumferentially closed filter sleeve. The filter sleeve may include a slit along its length to allow the at least one filter to be placed over the blood pump in a direction traverse to a central axis of the blood pump. The filter sleeve may have a mesh structure. The mesh may define smaller openings than the openings defined by the struts forming the housing. The mesh structure may include elongate slot-like apertures (361) separated by struts (362). At least a portion of the filter sleeve may include a shape-memory material. The filter sleeve may include a tapered portion at a first end (363), a second end (364), or both ends of the filter sleeve. In some embodiments, the filter(s) may be directly coupled to one or more struts (362) that are upstream (in the direction of the flow of blood) from the struts forming the housing. In some embodiments, the mesh may not be directly coupled to any struts. The mesh may be positioned upstream from the impeller. In some embodiments, the mesh may be positioned within an interior volume of space defined by the struts. In some embodiments, the mesh may be positioned external to the struts.

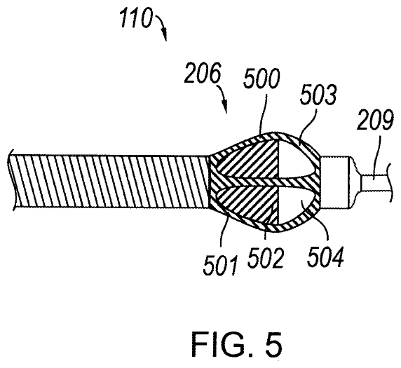

The blood flow section may include an expandable inlet (500). FIG. 5 illustrates an embodiment in which the blood flow inlet (206) may include an inflow funnel (502) in an expandible suction basket (501). The expandable inlet (500) may be made from a material able to restore itself, for example, or it is expanded by a balloon. The expandable inlet may include one or more struts (503). The funnel may be composed of a polymer. A polymer may be coupled to an inner or outer surface of the struts. In the expanded state, the suction basket (501) may have an outer diameter larger than that of the remainder of the blood flow section. The suction basket may have an outer diameter larger than that of the remainder of pump housing. Thus, the suction basket (501) may be expanded from its initial relatively small diameter (e.g., ≤4.5 mm) to a relatively larger diameter (e.g., >4.5 mm). Here, the inflow funnel (502) is shown as spanned from a flexible polymer screen that allows for a smooth inflow of blood into the funnel through openings (504) (which may be include a filter or mesh, not shown here for simplicity) forming an open distal end to allow blood to flow into the inflow funnel, and substantially increases the hydraulic capacity of the pump by reducing the hydraulic losses. The inflow funnel may define an external and/or internal surface of the blood flow section that are at least partially surface modified by the one or more fluorinated end groups and/or silicone end groups of an oligomer or copolymer.

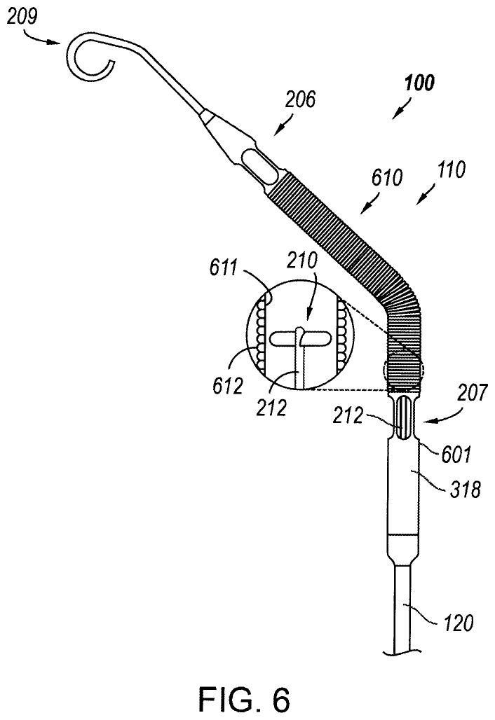

Referring to FIG. 6, in some embodiments, the blood flow section may include a cannula (610). The cannula may be coupled to a distal end (601) of a motor housing (318). The motor housing may define the blood flow outlet (207). The cannula may define an external surface (612) and/or internal surface (611) of the blood flow section that is at least partially surface modified by the one or more fluorinated end groups and/or silicone end groups of an oligomer or copolymer.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A blood pump, comprising:
a blood flow section operably coupled to a distal end of a catheter, the blood flow section configured to cause blood to flow into a blood flow inlet of the blood flow section, through the blood flow section, and out of a blood flow outlet;
wherein at least one external and/or internal surface of the blood flow section comprises at least a portion of a surface modified by one or more fluorinated end groups and/or silicone end groups of an oligomer or copolymer.

2. The blood pump of claim 1, wherein the at least a portion of a surface is modified by one or more fluorinated end groups of an oligomer.

3. The blood pump of claim 1, wherein the at least a portion of a surface is modified by one or more silicone end groups of a copolymer.

4. The blood pump of claim 1, wherein the oligomer or copolymer comprises a silicone, a polycarbonate, a polyurethane, a polyamide, a polyethylene, a polypropylene, a polysulfone, or a polyvinyl chloride.

5. The blood pump of claim 1, wherein a portion of both an internal surface and an external surface are modified by one or more fluorinated end groups and/or silicone end groups of an oligomer or copolymer.

6. The blood pump of claim 1, wherein all of an external and/or internal surface of the blood flow section are surface modified by one or more fluorinated end groups and/or silicone end groups of an oligomer or copolymer.

7. The blood pump of claim 1, wherein a first portion of an external and/or internal surface of the blood flow section is surface modified by one or more first fluorinated end groups and/or silicone end groups of a first oligomer or copolymer;
wherein a second portion of the external and/or internal surface of the blood flow section is surface modified by one or more second fluorinated end groups and/or silicone end groups of a second oligomer or copolymer where the first portion and second portions are different portions;
wherein:
the first fluorinated end groups and/or silicone end groups are different from the second fluorinated end groups and/or silicone end groups; and/or
the first oligomer or copolymer are different from the second oligomer or copolymer.

8. The blood pump of claim 1, wherein at least one portion of the blood flow section is expandable.

9. The blood pump of claim 8, wherein the blood flow section includes an expandable pump housing.

10. The blood pump of claim 9, wherein the expandable pump housing includes at least one strut.

11. The blood pump of claim 10, wherein the at least one strut is a helical strut configured to form a plurality of geometric apertures.

12. The blood pump of claim 10, wherein the at least one strut is a polymer.

13. The blood pump of claim 10, wherein the at least one strut is coupled to a polymer.

14. The blood pump of claim 13, wherein the polymer is coupled to an inner surface of the at least one strut.

15. (canceled)

16. The blood pump of claim 12, wherein the polymer defines the at least one external and/or internal surface of the blood flow section that are at least partially surface modified by the one or more fluorinated end groups and/or silicone end groups of an oligomer or copolymer.

17. The blood pump of claim 12, wherein the polymer is disposed on at least one intermediate portion of the expandable pump housing, the intermediate portion starting a distance d1>0 from a distal end of the expandable pump housing and a distance d2>0 from a proximal end of the expandable pump housing.

18. The blood pump of claim 17, wherein the polymer forms an inner liner preventing blood from flowing radially through the blood flow section in the intermediate portion.

19. (canceled)

20. The blood pump of claim 8, wherein the blood flow section includes an expandable inlet.

21-25. (canceled)

26. The blood pump of claim 1, wherein the blood flow section includes a motor housing coupled to a pump housing, the pump housing defining the blood flow inlet and a pump housing outlet.

27-29. (canceled)

30. The blood pump of claim 1, wherein the blood flow section further comprises at least one filter placed over the blood flow inlet as a filter sleeve, wherein the filter sleeve filters the blood entering the blood flow inlet.

31-42. (canceled)
